(19) United States
(12) Patent Application Publication
(10) Pub. No.: US 2016/0158052 A1
(43) Pub. Date: Jun. 9, 2016

(54) Medical Device and Application Thereof

(71) Applicant: Ping WAN, Shanghai (CN)

(72) Inventor: Ping WAN, Shanghai (CN)

(21) Appl. No.: 14/906,239

(22) PCT Filed: Jun. 12, 2014
(86) PCT No.: PCT/CN2014/079705

§ 371 Date: Jan. 19, 2016

(30) Foreign Application Priority Data
Jul. 18, 2013 (CN) 201310301366.8

(51) Int. Cl. A61F 5/00

(52) U.S. Cl. CPC A61F 5/0076

(57) ABSTRACT

A duodenal internal covering membrane is disclosed herein. The covering membrane is made of a biocompatible biodegradable or non-biodegradable material and/or hydrophobic material and mainly comprises an elastic ampulla and a tubular portion. The ampulla contains biocompatible bionic microarray adhesive piece capable of realizing strong adhesion through a force exerting direction without pricking into an intestinal tissue and capable of being easily detached and recovered. The adhesive piece has good stability, strong adaptability to material and appearance, good self-cleaning property, no injuries and pollution to the intestinal tissue and can be adhered and detached repeatedly. The adhesive piece can realize mutual support with other parts in the functions. When contents in the intestinal duct move, because there is no traction force which is nearly perpendicular, the internal covering membrane cannot be detached. When the duodenal bulb expands, as the internal covering membrane has no opposite traction force which is nearly perpendicular, the internal covering membrane cannot be detached either. When recovery is performed, the detachment and recovery can be easily realized by force exerting which is nearly perpendicular. The duodenal internal covering membrane can be prepared into a medical device for preventing and treating obesity and diabetes without injuring the intestinal tissue.

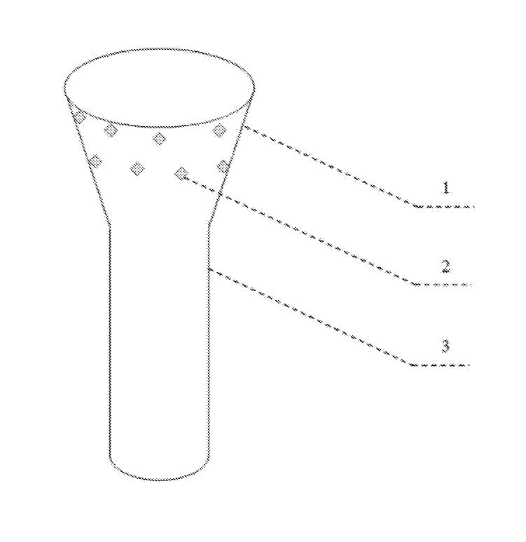

### BACKGROUND OF THE INVENTION

1. Field of the Invention

This invention relates to a medical device placed in the digestive tract and, more particularly, to a duodenal internal covering membrane for preventing and treating obesity and diabetes without injuring an intestinal tissue.

2. Background of the Invention

Gastric bypass surgery can treat obesity. Recently, it was found that, for an obese patient undergoing the surgery, not only the body weight was significantly decreased, but also the type 2 diabetes complicated by obesity was alleviated (Chinese Journal of Diabetes, 2011, 3(3): 205-208): after the surgery, the blood sugar problem could be solved without injecting insulin or taking multiple medicaments, and hypertension, obesity, blood lipid disorders and other diabetic complications could be further obviously improved. According to the analysis of 22094 cases of the gastric bypass surgery: 84% of the patients with the type 2 diabetes completely reversed after the surgery, and most of the patients stopped the treatment with oral medicaments or insulin before leaving the hospitals (Chinese medical science, 2011,1 (21): 3-5). Foreign countries including the US government have been actively promoting the development of the surgery. In March, 2011, in the 2^{nd} international conference of interventional treatment of type 2 diabetes held in New York, US, the International Diabetes Federation (IDF) firstly issued a statement that, it was considered that the gastric bypass surgery can be used for treating the obese patients with the type 2 diabetes and could reduce the occurrence and the development of chronic complications of the diabetes (Chinese medical science, 2011,1 (22): 1-2), and if the surgery was performed early, the serious complications of the diabetes could also be prevented (Diabetes World: Abstracts Journal, 2011, 10: 51).

However, the “gastric bypass surgery” has clinical risks, such as death, intestinal obstruction, anastomotic leakage, pulmonary embolism, deep vein thrombosis, portal vein injury, diseases of respiratory system and the like (Chinese Journal of Diabetes, 2011, 3 (3): 205-208). Thus, the way of placing the duodenal internal covering membrane in the duodenum in vivo for preventing and treating the obesity and the diabetes has application significance for replacing the “gastric bypass surgery”.

In the invention patent of “duodenal sleeve and conveyor thereof” (filing date: Apr. 9, 2010 and issue date: Jan. 11, 2012) in the prior art, although a disposable static “expanded” metal framework which covers an outer sleeve and only depends on “a memory alloy” is “fully close to” the intestinal wall and “the metal framework of a duodenal bulb cavity section” which is “bowl and funnel-shaped” is “jointed with the duodenal bulb cavity”, the duodenum is mobile, particularly when the gastric pylorus expands and draws the duodenal bulb which is just below the gastric pylorus to expand on the upper edge of the duodenal bulb, the metal framework is difficult to perform elastic expansion and accordingly move to the distal end of the duodenum, and when the gastric pylorus retracts, the metal framework may perform reverse reset (the mucosa of the duodenal bulb is relatively thin and is the susceptible site of ulcers; and if the process is repeated in such a way, the mucosa is liable to injuries, and a muscular wall stretch receptor is liable to stimulation, thereby being liable to inducing nausea or/and vomiting [Zhou Lv, Ke Meiyun: “Gastrointestinal dynamics: base and clinic” page 117] or perform incarceration at the distal end of the duodenal bulb by flexible intestinal compensative expansion. A metal framework in the invention patent of “device for delaying gastric emptying and regulating metabolism of intestines and pancreas (filing date: Feb. 22, 2012 and application publication date: Jul. 11, 2012) in the prior art is also like this.

In order to solve the problem of fixation, according to the utility model patent of “duodenum-jejunum built-in sleeve” (filing date: Dec. 6, 2010 and issue date: Sep. 28, 2011) in the prior art, a hollow metal tube is made into a spike fixing claw, which is “sleeved on metal wires of an annular stent” and is tightly “fixed”, but it must be pricked into the intestinal mucosa, thereby causing direct injuries to an intestinal tissue. Then, in order to solve the removal problem in the future, according to the prior art (utility mode patent of “duodenum-jejunum built-in sleeve”), a tightening thread is further designed, the tightening thread is “placed at the top of the annular stent”, and “can be wound around an upper opening one circle or multiple circles”, but by implanting the sleeve in vivo, particularly under the situation of only considering that the spike fixing claw made of the hollow metal tube on the static annular stent with “simplicity and convenience in manufacturing and tightening performance” is pierced into the inner wall of the duodenal bulb cavity, and along with the gastrointestinal motion, looseness, exudation and adhesion are repeated continuously. According to the prior art (utility mode patent of “duodenum-jejunum built-in sleeve”), the material of a flexible tube only considers “smooth surface, softness and compactness”, the adaptive elastic expansion and contraction of the annular stent to the movements of the duodenal bulb and the adoption of the elastic material for preparing the flexible tube are not involved in the patent; and the tightening thread without elasticity which is “fixed” at “the top of the annular stent” on the upper edge of the duodenal bulb even limits the compliance of the annular stent to the movements of the duodenal bulb. Compared with the invention patent of “duodenal sleeve and conveyor thereof” in the prior art, in the prior art (utility model patent of “duodenum-jejunum built-in sleeve”), as the annular stent and the spike fixing claw thereof are only positioned in the duodenal bulb, although the duodenal papilla at the common opening of the bile duct and the pancreatic duct at the lower end of the duodenal descending part is not blocked, but if they are placed on the upper edge of the duodenal bulb, when the gastric pylorus expands or retracts to draw the duodenal bulb which is just below the gastric pylorus to move (Zhou Lv, Ke Meiyun: “Gastrointestinal dynamics: base and clinic” pages 381, 520 and 522), the annular stent and the spike fixing claw thereof are bound to hinder the movements of the duodenal bulb, particularly when the gastric pylorus expands to draw the duodenal bulb to move, the annular stent and the spike fixing claw thereof cannot accordingly expand, the spike fixing claw pierced into the mucosa of the duodenal bulb also inwards and centripetally pulls the mucosal tissue of the duodenal bulb to be opposite to the movements of muscle tissue and other tissues below the mucosa of the duodenal bulb, which expand outwards and centrifugally as a whole, and obviously, when the gastric pylorus expands or retracts to draw the duodenal bulb which is just below the gastric pylorus to move, such annular stent and the spike fixing claw thereof which cannot change or change a little accordingly can cause injuries to the duodenal bulb tissue; and if they are placed on the lower edge of the duodenal bulb, although the drawing of the lower edge of the duodenal bulb by expansion or retraction of the gastric pylorus is less than the drawing of the upper edge of the duodenal bulb, even the injuries to the mucosa of the duodenal bulb caused by the annular stent and the spike fixing claw thereof are ignored, the original effect of blocking the duodenal bulb also disappears accordingly.

Similarly, a nitinol anchoring part in the invention patent of “duodenum sleeve and preparation method thereof” (filing date: May 10, 2012 and application publication date: Oct. 3, 2012) in the prior art must also be pricked into the intestinal mucosa, thereby causing direct injuries to the intestinal tissue.

### BRIEF SUMMARY OF THE INVENTION

### Technical Problems to be Solved by the Invention

As for the invention patent of “duodenal sleeve and conveyor thereof” (filing date: Apr. 9, 2010 and issue date: Jan. 11, 2012) in the prior art and the invention patent of “device for delaying gastric emptying and regulating metabolism of intestines and pancreas (filing date: Feb. 22, 2012 and application publication date: Jul. 11, 2012) in the prior art, a metal framework is easy to cause injuries to the intestinal mucosa, easy to stimulate the intestinal wall and further induce nausea or/and vomiting, and as for the utility model patent of “duodenum-jejunum built-in sleeve” (filing date: Dec. 6, 2010 and issue date: Sep. 28, 2011) in the prior art and the invention patent of “duodenum sleeve and preparation method thereof” (filing date: May 10, 2012 and application publication date: Oct. 3, 2012) in the prior art, when fixation is performed, the sleeve must be pricked into the intestinal mucosa, thereby causing direct injuries to an intestinal tissue. An ampulla elastic membrane and bionic microarray adhesive piece adhered thereon of the invention have small area, low thickness and strong grasping force without pricking into the mucosa, the force exerting angle of flow of intestinal contents is difficult to realize detachment, the force exerting angle of a duct of endoscopic forceps can easily realize detachment and mounting, and adhesion and detachment can be performed repeatedly without injuring the intestinal tissue.

In the invention patent of “duodenal sleeve and conveyor thereof” (filing date: Apr. 9, 2010 and issue date: Jan. 11, 2012) in the prior art, for a disposable static “expanded” and “bowl and funnel-shaped” “ metal framework of a duodenal bulb cavity section” which covers an outer sleeve and only depends on “a memory alloy”, when the gastric pylorus expands and draws the duodenal bulb which is just below the gastric pylorus to expand, the metal framework is difficult to perform elastic expansion and accordingly move to the distal end of the duodenum, and when the gastric pylorus retracts, the metal framework may perform reverse reset (mucosa is liable to injuries) or perform incarceration at the distal end of the duodenal bulb by flexible intestinal compensative expansion. The upper section of a duodenal internal covering membrane of the invention can be a wavy, V-shaped, trapezoidal or crenellated ampulla elastic membrane, the outer surface of the ampulla elastic membrane is adhered with bionic microarray adhesive piece, and the whole can perform telescopic or elastic movements by complying with the motion of the duodenum and the bulb, thereby solving this problem.

In the invention patent of “duodenal sleeve and conveyor thereof” (filing date: Apr. 9, 2010 and issue date: Jan. 11, 2012) in the prior art, the back section of the metal framework duodenal bulb blocks the duodenal papilla at a common opening of the bile duct and the pancreatic duct at the lower end of the duodenal descending part. In the invention, the ampulla of the duodenal internal covering membrane are fixed by the bionic microarray adhesive piece and does not block the duodenal papilla at the common opening of the bile duct and the pancreatic duct at the lower end of the duodenal descending part, thereby solving this problem.

In order to solve the problem of fixation, according to the utility model patent of “duodenum-jejunum built-in sleeve” (filing date: Dec. 6, 2010 and issue date: Sep. 28, 2011) in the prior art, a spike fixing claw which is made of a hollow metal tube on a static annular stent which only considers “simplicity and convenience in manufacturing and tightening performance) is pierced into the inner wall of the duodenal bulb cavity. According to the prior art (utility mode patent of “duodenum-jejunum built-in sleeve”), the material of a flexible tube only considers “smooth surface, softness and compactness”, the adaptive elastic expansion and contraction of the annular stent to the movements of the duodenal bulb and the adoption of the elastic material for preparing the flexible tube are not involved in the patent; and the tightening thread without elasticity which is “fixed” at “the top of the annular stent” on the upper edge of the duodenal bulb even limits the compliance of the annular stent to the movements of the duodenal bulb. If the annular stent and the spike fixing claw thereof are placed on the upper edge of the duodenal bulb, when the gastric pylorus expands or retracts to draw the duodenal bulb which is just below the gastric pylorus to move, the annular stent and the spike fixing claw thereof are bound to hinder the movements of the duodenal bulb, particularly when the gastric pylorus expands to draw the duodenal bulb to move, the annular stent and the spike fixing claw thereof cannot accordingly expand, the spike fixing claw pierced into the mucosa of the duodenal bulb also inwards and centripetally pulls the mucosal tissue of the duodenal bulb to be opposite to the movements of muscle tissue and other tissues below the mucosa of the duodenal bulb, which expand outwards and centrifugally as a whole, and obviously, when the gastric pylorus expands or retracts to draw the duodenal bulb which is just below the gastric pylorus to move, such annular stent and the spike fixing claw thereof which cannot change or change a little accordingly can cause injuries to the duodenal bulb; and if the annular stent and the spike fixing claw thereof are placed on the lower edge of the duodenal bulb, although the drawing of the lower edge of the duodenal bulb by expansion or retraction of the gastric pylorus is less than the drawing of the upper edge of the duodenal bulb, even the injuries to the mucosa of the duodenal bulb caused by the annular stent and the spike fixing claw thereof are ignored, the original effect of blocking the duodenal bulb also disappears accordingly. The ampulla elastic membrane and the bionic microarray adhesive piece adhered thereon of the invention can perform telescopic or elastic motion by complying with the motion of the duodenum and the bulb as a whole, thereby not only preventing causing injuries to the duodenal bulb tissue, but also blocking the duodenal bulb. Thus, this problem can be solved. Secondly, the ampulla elastic membrane and the bionic microarray adhesive piece adhered thereon of the invention do not affect the compliance to the motion of the duodenum and the bulb, and when the internal covering membrane is recovered, the easy detachment can be realized by the force which is nearly perpendicular via a duct of endoscopic forceps, thereby recovering the duodenal internal covering membrane of the invention.

### Technical Scheme of the Invention

This invention provides a duodenal internal covering membrane. All the parts of the duodenal internal covering membrane can be made of biocompatible biodegradable or non-biodegradable materials or/and strongly hydrophobic materials.

The duodenal internal covering membrane can include an ampulla and a tubular portion, wherein the ampulla is positioned in the duodenal bulb and the tubular portion can extend to the jejunum.

The diameter, the length and the thickness of the tubular portion can be matched with the duodenum and the jejunum in each person of different people groups. Preferably, the diameter is 10-60 mm, the length is matched with the duodenum and can extend to one section of the jejunum following the duodenum, the length is 80-700 mm, and the thickness of the internal covering membrane of the tubular portion is 0.005 mm-1 mm.

The ampulla is a flared part following the tubular portion. Preferably, the ampulla can be cylindrical, spherical and waist drum-shaped. Preferably, the thickness of the internal covering membrane of the ampulla is 0.005 mm-1 mm, the height is 6 mm-100 mm, the flared part following the tubular portion forms a progressive open acute angle, which is preferably 5° C.-65° C., and the thickness, the height and the angle can be matched with different people groups.

Preferably, the preparation of the ampulla and the tubular portion of the duodenal internal covering membrane can adopt electrostatic spinning, electrostatic spraying, tape casting, laminating, a micro/nano-process or/and an anti-sticking process, and the material can be a biocompatible degradable or biocompatible non-degradable or/or/and strongly hydrophobic or other known materials and a combination thereof.

Preferably, the upper edge of the ampulla of the duodenal internal covering membrane can be a wavy, V-shaped, trapezoidal or crenellated elastic membrane. Preferably, the bionic microarray adhesive piece is made of a biocompatible biodegradable or non-biodegradable material or/and hydrophobic material, which can be selected from silicon rubber, polyurethane, multi-wall carbon nanotubes, polyester resin, polyimide, artificial rubber, epoxy resin, polydimethylsiloxane, polystyrene, polytetrafluoroethylene, Teflon, polydimethylsiloxane, poly-p-xylene, polyurethane, ethylene terephthalate, polymethylmethacrylate and the like or a combination thereof and other known suitable materials, the shape can be circular, oval, trapezoidal, square, triangular, cylindrical, rhombic, special-shaped and the like or a combination thereof, the size can be 1 nm² or above or a combination thereof, and the top end of adhesion fiber fine hair can be a curved (shovel-like) or flat pressing head-like or circular pressing head-like or layered structure or other shapes and structures and a combination thereof. Preferably, the bionic microarray adhesive piece can be adhered to the ampulla of the internal covering membrane by suturing, adhering, anchoring, weaving, hooking and clamping, riveting, thermoplastic treatment, freezing, gas pressing, electrostatic treatment and the like or/and a combination thereof or other known methods and the like and a combination thereof, the precise arrangement can be circular, oval, trapezoidal, square, triangular, cylindrical, rhombic, special-shaped and the like or a combination thereof, one or more than one row can be adopted, the bionic microarray adhesive piece can be arranged closely or separately or in other manners or arranged in a combination manner, and an adhesive can be biocompatible polyurethane, polyurethane, silicone resin, fluorinated ethylene propylene and the like or a combination thereof or other known materials and a combination thereof.

Preferably, the preparation of the bionic microarray adhesive piece adhered on the ampulla of the duodenal internal covering membrane can adopt an inductive coupling plasma (ICP) deep etching technology in a micro-electro-mechanical system (MEMS) to etch an erective microarray template on a silicon chip, cast polydimethoxysiloxane (PDMS) onto a silicon template pillar array, cure and then strip for demolding to obtain a polydimethoxysiloxane (PDMS) microarray hole template, cast liquid polyurethane or/and other biocompatible material onto the polydimethoxysiloxane (PDMS) micro-hole template, cure and demold to obtain a polyurethane bionic adhesion microarray. The invention does not exclude the use of other substances and other methods for preparing the adhesion microarray.

Preferably, a mussel-inspired adhesive protein polymer-dopamine-methacrylamide/methoxyethyl acrylate copolymer (P(DMA-co-MEA)) is synthesized, and other known methods can also be used; and the synthesized dopamine-containing copolymer is dissolved in a dichloromethane solution, the polyurethane microarray is immersed in the solution, and a layer of dopamine-containing copolymer is modified on the outer surface of the polyurethane microarray. The invention does not exclude other substances (including modifying substances and modified substances) which can form strong adhesive force in drying conditions and also have strong adhesive force in water and other preparation methods. Preferably, the bionic microarray adhesive piece has suitable contact surfaces, and the diameter-length ratio of the fine hair and the spacing interval between the fine hair are controlled for preventing mutual adhesion; and preferably, the diameter-length ratio of the fine hair is 0.1-5: 20, the length is 0.1-200 μm and the spacing interval between the fine hair is 0.1-30.0 μm.

In the preparation process of the bionic microarray adhesive piece, an atomic force microscope etching method can also be used as below: using the tapered pointed end of a probe of an atomic force microscope to etch micro-holes on the surface of smooth paraffin wax, casting a liquid raw material into the holes, placing for cooling, removing the paraffin wax, and demolding, thereby forming micro-protuberances which are similar to a fine forked structure on gecko's setae and have the similar size on the surface of the polymer.

In the preparation process of the bionic microarray adhesive piece, an alumina template hole injection forming method can also be used as below: putting an aluminum foil into an acidic electrolyte, and performing anodic oxidation to form an alumina plate with holes, wherein the aperture and the spacing interval between the holes can be regulated and controlled by oxidation voltage and an acidic solution. Other mold injection methods can also be used.

In the preparation process of the bionic microarray adhesive piece, an electrostatic induction etching method can also be used as below: using a solution spin-coating method to prepare a layer of polymer thin membrane on a smooth silicon chip to serve as a lower electrode, taking another silicon chip as an upper electrode, retaining an air gap between the surface of the polymer and the upper electrode, heating the polymer to be above glass transition temperature, applying direct current voltage to a capacitor, producing electric field intensity, forming a regular micro-structure and cooling to room temperature to obtain the corresponding polymer. If the upper electrode itself has the micro-structure, the polymer can accurately reproduce the same protuberant micro-structure.

In the preparation process of the bionic microarray adhesive piece, an inductive coupling plasma etching technology can also be used as below: passivating and etching a silicon template with special gas, wherein a CRYO process and a BOSCH process can be adopted. The CRYO process is as follows: synchronously passivating and etching below −100° C., wherein gas can be SF₆/O₂. The BOSCH process is as follows: separately etching and passivating at normal temperature, wherein etching gas can be SF₆, and passivating gas can be C₄/F₈.

In the preparation process of the bionic microarray adhesive piece, a photolithography technology (electron beam projection lithography, nano-imprint lithography and the like) can be used as below: drawing a mask which is tens or hundreds of times bigger than the actual size artificially or by a computer, scaling down to form an actual working template, attaching the template on a silicon substrate, and etching a bionic array shape which is the same with the template on the silicon substrate by a photon beam transmitting the template.

In the preparation process of the bionic microarray adhesive piece, array nano-carbon tubes can also be used as below: decomposing gas containing a carbon element at high temperature by a chemical vapor deposition method, and enabling decomposed carbon atoms to generate an ordered carbon nanotube array directionally under the action of a catalyst. The chemical vapor deposition method can be a thermal chemical vapor deposition (TCVD) method, a plasma enhanced chemical vapor deposition (PECVD) method, a floating catalyst chemical vapor deposition (FCCVD) method and the like.

In the preparation process of the bionic microarray adhesive piece, a reactive plasma dry etching method can also be used as below: preparing a layer of polymer thin membrane with micro-scale thickness on a silicon chip, etching the aluminum membrane by an electron beam to form a micro-structure array, and performing dry etching by using oxygen plasma by means of great difference in etching rate of oxygen plasma between alumina and the polymer to transfer and reproduce the micro-structure on the aluminum membrane onto the polymer thin membrane.

In the preparation process of the bionic microarray adhesive piece, a soft etching method, a directed self-assembly method based on growth of micro/nano fine hair and the like can be used.

Preferably, the tubular portion or/and the ampulla of the internal covering membrane can be added with one or more longitudinal or oblique or crisscross or spiral or special-shaped ribs or a combination thereof by suturing, adhering, anchoring, weaving, hooking and clamping, riveting, thermoplastic treatment, freezing, gas pressing, electrostatic treatment and the like or/and a combination thereof or other known methods, and the ribs can reinforce, support, expand the internal covering membrane and prevent distortion thereof, and have other functions of realizing coordination with the internal covering membrane and realizing mutual support with the functions of the internal covering membrane.

Preferably, the ampulla and the tubular portion of the duodenal internal covering membrane can be closed up or folded into the shape of a ball, cylinder, capsule or spindle together in vitro, and the folding way can be as follows: the internal covering membrane is folded or curled or covered from the distal end to the proximal end and then the ampulla is centripetally turned inwards.

Preferably, the duodenal internal covering membrane can be sent into the duodenum via the upper digestive tract under the assistance of an endoscope and X-ray fluoroscopy or other medical or/and biological observation equipment and instruments, when the covering membrane is arranged, a multi-claw device (the number of claws can be matched with the precise arrangement of the bionic microarray adhesive piece) can perform centrifugal distraction on the ampulla which is tuned inwards along the center via a duct of endoscopic forceps or/and other devices, further outwards turn and reset the ampulla which is tuned inwards, position it in the duodenal bulb, adhere, and then softly push the distal end of the duodenal internal covering membrane to a target position by the device or/and gas or/and water or/and gravity or/and other methods. If a memory material is adopted, the covering membrane can be gradually spread out at memory temperature in the intestine, the ampulla of the internal covering membrane is placed at the upper part of the duodenum, and the lower edge of the ampulla is arranged on the side of the duodenal papilla and the auxiliary papilla (minor papilla) near the gastric pylorus and does not hinder liquid from the bile duct and the pancreatic duct from entering the intestinal cavity. The tubular portion of the internal covering membrane is positioned at the duodenal descending part, the horizontal part and the ascending part following the upper part of the duodenum, and the extended tubular portion is positioned in the jejunum section following the ascending part of the duodenum. When contents in the intestinal duct move, due to the absence of traction force which is nearly perpendicular, the duodenal internal covering membrane cannot be detached; and when the duodenal bulb expands, as the duodenal internal covering membrane has no opposite traction force which is nearly perpendicular, the duodenal internal covering membrane cannot be detached.

Preferably, when the duodenal internal covering membrane is recovered, a multi-claw device (the number of claws can be matched with the precise arrangement of the bionic microarray adhesive piece) can be inserted from the upper edge of the ampulla via the duct of the endoscopic forceps or/and other devices and easily realize detachment and recovery accordingly at the part forming an included angle of about 90° with the upper edge of the ampulla by centripetal traction force which is near to the perpendicular direction, thereby avoiding avulsion and other injuries to the intestinal tissue. After detachment, the upper edge of the ampulla is turned inwards immediately, then the detached bionic microarray adhesive piece is accordingly adhered with other parts of the ampulla itself, and then the duodenal internal covering membrane can be easily taken out and recovered.

Preferably, the duodenal internal covering membrane and the bionic microarray adhesive piece thereof are soft, smooth, elastic and good in tissue compatibility, and have no acute systemic reaction, no chronic systemic reaction, no acute local reaction and no chronic local reaction.

The duodenal internal covering membrane diverts chyme and bile and pancreatic juice in vivo, avoids direct digestion, absorption and metabolism of gastric effluent in the duodenum, and can be prepared into a medical device for preventing and treating obesity and diabetes without injuring the intestinal tissue.

The length, the thickness, the elasticity and the shape of each part, the diameter-length ratio of the fine hair, the length of the fine hair, the diameter of the fine hair, the spacing interval between the fine hair and other parameters of the duodenal internal covering membrane are reference values, and can be specially designed according to needs in actual manufacturing.

### Benefits of the Invention

The invention provides a duodenal internal covering membrane, wherein the internal covering membrane is made of a biocompatible biodegradable or non-biodegradable material and/or strongly hydrophobic material and mainly comprises an elastic ampulla and a tubular portion, wherein the ampulla is positioned in the duodenal bulb, the tubular portion can extend to the jejunum, the ampulla contains biocompatible bionic microarray adhesive piece which can realize strong adhesion through the force exerting direction without pricking into an intestinal tissue and can be easily detached and recovered, the upper section of the duodenal internal covering membrane can be a wavy, V-shaped, trapezoidal or crenellated ampulla elastic membrane, the ampulla attached with the bionic microarray adhesive piece can perform telescopic or elastic motion by complying with the motion of the duodenum and the bulb as a whole, and the ampulla and the tubular portion can be closed up or folded into the shape of a ball, cylinder, capsule or spindle together in vitro. The bionic microarray adhesive piece has small area, low thickness and strong grasping force without pricking into the mucosa, the force exerting angle of flow of intestinal contents is difficult to realize detachment, the force exerting angle of a duct of endoscopic forceps can easily realize detachment, recovery and mounting, and adhesion and detachment can be performed repeatedly. The bionic microarray adhesive piece has the advantages of great adhesive force, good stability, strong adaptability to material and appearance, good self-cleaning property, no injuries and pollution to the intestinal tissue and the like, can realize mutual support with other parts in the functions and can be prepared into a medical device for preventing and treating obesity and diabetes without injuring the intestinal tissue.

Compared with the invention patent of “duodenal sleeve and conveyor thereof” (filing date: Apr. 9, 2010 and issue date: Jan. 11, 2012) in the prior art and the invention patent of “device for delaying gastric emptying and regulating metabolism of intestines and pancreas (filing date: Feb. 22, 2012 and application publication date: Jul. 11, 2012) in the prior art, the duodenal internal covering membrane of the invention can perform telescopic or elastic motion by complying with the motion of the duodenum and the bulb as a whole, thereby preventing the injuries to the duodenum tissue either in a static state or a dynamic state, avoiding the incarceration in the flexible intestinal duct, and not hindering the effluent of the bile duct and the pancreatic duct from entering the intestinal tube. Compared with the utility model patent of “duodenum-jejunum built-in sleeve” (filing date: Dec. 6, 2010 and issue date: Sep. 28, 2011) in the prior art, the ampulla elastic membrane and the bionic microarray adhesive piece thereof of the invention can perform telescopic or elastic motion by complying with the motion of the duodenum and the bulb as a whole, thereby not only preventing the injuries to the tissue of the duodenal bulb, but also blocking the duodenal bulb. Compared with the utility model patent of “duodenum-jejunum built-in sleeve” (filing date: Dec. 6, 2010 and issue date: Sep. 28, 2011) in the prior art, the invention patent of “duodenum sleeve and preparation method thereof” (filing date: May 10, 2012 and application publication date: Oct. 3, 2012) in the prior art, the invention patent of “duodenal sleeve and conveyor thereof” (filing date: Apr. 9, 2010 and issue date: Jan. 11, 2012) and the utility model patent of “duodenum-jejunum built-in sleeve” (filing date: Dec. 6, 2010 and issue date: Sep. 28, 2011), the duodenal internal covering membrane and the bionic microarray adhesive piece of the ampulla elastic membrane thereof of the invention have the advantages of great convenience in detachment and recovery, no injuries to the intestinal tissue and repeated use.

### BRIEF DESCRIPTION OF THE DRAWINGS

The figure is a structural schematic diagram.

In the figure, the parts or the sites represented by the reference numbers are as follows: 1-elastic ampulla; 2-bionic microarray adhesive piece; and 3-tubular portion.

### DETAILED DESCRIPTION OF THE INVENTION

This invention is further described below in conjunction with the accompanying drawing and the specific examples.

As shown in the drawing, the invention provides a duodenal internal covering membrane. All the parts of the duodenal internal covering membrane can be made of biocompatible biodegradable or non-biodegradable materials or/and strongly hydrophobic materials. The duodenal internal covering membrane can be divided into an ampulla 1 and a tubular portion 3, wherein the ampulla 1 is positioned in the duodenal bulb, the tubular portion 3 can extend to the jejunum and bionic microarray adhesive piece 2 is adhered on the outer side of the ampulla 1.

The diameter, the length and the thickness of the tubular portion 3 can be matched with the duodenum and the jejunum in each person of different people groups. Preferably, the diameter is 10-60 mm, the length is matched with the duodenum and can extend to one section of the jejunum following the duodenum, the length is 80-700 mm, and the thickness of the internal covering membrane of the tubular portion is 0.005 mm-1 mm. The ampulla 1 is a flared part following the tubular portion 3. Preferably, the ampulla 1 can be cylindrical, spherical and waist drum-shaped. Preferably, the thickness of the internal covering membrane of the ampulla 1 is 0.005 mm-1 mm, the height is 6 mm-100 mm, the flared part following the tubular portion 3 forms a progressive open acute angle, which is preferably 5° C.-65° C., and the thickness, the height and the angle can be matched with different people groups.

Preferably, the upper edge of the ampulla 1 can be a wavy, V-shaped, trapezoidal or crenellated elastic membrane.

Preferably, the bionic microarray adhesive piece 2 is made of a biocompatible biodegradable or non-biodegradable material or/and hydrophobic material, which can be selected from silicon rubber, polyurethane, multi-wall carbon nanotubes, polyester resin, polyimide, artificial rubber, epoxy resin, polydimethylsiloxane, polystyrene, polytetrafluoroethylene, Teflon, polydimethylsiloxane, poly-p-xylene, polyurethane, ethylene terephthalate, polymethylmethacrylate and the like or a combination and other know suitable materials, the shape can be circular, oval, trapezoidal, square, triangular, cylindrical, rhombic, special-shaped and the like or a combination thereof, the size can be 1 nm² or above or a combination thereof, and the top end of adhesion fiber fine hair can be a curved (shovel-like) or flat pressing head-like or circular pressing head-like or layered structure or other shapes and structures and a combination thereof. Preferably, the bionic microarray adhesive piece 2 can be adhered to the ampulla 1 of the internal covering membrane by suturing, adhering, anchoring, weaving, hooking and clamping, riveting, thermoplastic treatment, freezing, gas pressing, electrostatic treatment and the like or/and a combination thereof or other known methods and the like and a combination thereof, the precise arrangement can be circular, oval, trapezoidal, square, triangular, cylindrical, rhombic, special-shaped and the like or a combination thereof, one or more than one row can be adopted, the bionic microarray adhesive piece 2 can be arranged closely or separately or in other manners or arranged in a combination manner, and an adhesive can be biocompatible polyurethane, polyurethane, silicone resin, fluorinated ethylene propylene and the like or a combination thereof or other known materials and a combination thereof.

Preferably, the preparation of the bionic microarray adhesive piece 2 adhered on the ampulla 1 of the duodenal internal covering membrane can adopt an inductive coupling plasma (ICP) deep etching technology in a micro-electro-mechanical system (MEMS) to etch an erective microarray template on a silicon chip, cast polydimethoxysiloxane (PDMS) onto a silicon template pillar array, cure and then strip for demolding to obtain a polydimethoxysiloxane (PDMS) microarray hole template, cast liquid polyurethane or/and other biocompatible material onto the polydimethoxysiloxane (PDMS) micro-hole template, cure and demold to obtain a polyurethane bionic adhesion microarray. The invention does not exclude the use of other substances and other methods for preparing the adhesion microarray.

Preferably, a mussel-inspired adhesive protein polymer-dopamine-methacrylamide/methoxyethyl acrylate copolymer (P(DMA-co-MEA)) is synthesized, and other known methods can also be used; and the synthesized dopamine-containing copolymer is dissolved in a dichloromethane solution, the polyurethane microarray is immersed in the solution, and a layer of dopamine-containing copolymer is modified on the outer surface of the polyurethane microarray. The invention does not exclude other substances (including modifying substances and modified substances) which can form strong adhesive force in drying conditions and also have strong adhesive force in water and other preparation methods. Preferably, the bionic microarray adhesive piece 2 have suitable contact surfaces, and the diameter-length ratio of the fine hair and the spacing interval between the fine hair are controlled for preventing mutual adhesion; and preferably, the diameter-length ratio of the fine hair is 0.1-5: 20, the length is 0.1-200 μm and the spacing interval between the fine hair is 0.1-30.0 μm.

In the preparation process of the bionic microarray adhesive piece 2, an atomic force microscope etching method can also be used as below: using the tapered pointed end of a probe of an atomic force microscope to etch micro-holes on the surface of smooth paraffin wax, casting a liquid raw material into the holes, placing for cooling, removing the paraffin wax, and demolding, thereby forming micro-protuberances which are similar to a fine forked structure on gecko's setae and have the similar size on the surface of the polymer.

In the preparation process of the bionic microarray adhesive piece 2, an alumina template hole injection forming method can also be used as below: putting an aluminum foil into an acidic electrolyte, and performing anodic oxidation to form an alumina plate with holes, wherein the aperture and the spacing interval between the holes can be regulated and controlled by oxidation voltage and an acidic solution. Other mold injection methods can also be used.

In the preparation process of the bionic microarray adhesive piece 2, an electrostatic induction etching method can also be used as below: using a solution spin-coating method to prepare a layer of polymer thin membrane on a smooth silicon chip to serve as a lower electrode, taking another silicon chip as an upper electrode, retaining an air gap between the surface of the polymer and the upper electrode, heating the polymer to be glass transition temperature, applying direct current voltage to a capacitor, producing electric field intensity, forming a regular micro-structure and cooling to room temperature to obtain the corresponding polymer. If the upper electrode itself has the micro-structure, the polymer can accurately reproduce the same protuberant micro-structure.

In the preparation process of the bionic microarray adhesive piece 2, an inductive coupling plasma etching technology can also be used as below: passivating and etching a silicon template with special gas, wherein a CRYO process and a BOSCH process can be adopted. The CRYO process is as follows: synchronously passivating and etching below −100° C., wherein gas can be SF₆/O₂. The BOSCH process is as follows: separately etching and passivating at normal temperature, wherein etching gas can be SF₆, and passivating gas can be C₄/F₈.

In the preparation process of the bionic microarray adhesive piece 2, a photolithography technology (electron beam projection lithography, nano-imprint lithography and the like) can be used as below: drawing a mask which is tens or hundreds of times bigger than the actual size artificially or by a computer, scaling down to form an actual working template, attaching the template on a silicon substrate, and etching a bionic array shape which is the same with the template on the silicon substrate by a photon beam transmitting the template.

In the preparation process of the bionic microarray adhesive piece 2, array nano-carbon tubes can also be used as below: decomposing gas containing a carbon element at high temperature by a chemical vapor deposition method, and enabling decomposed carbon atoms to generate an ordered carbon nanotube array directionally under the action of a catalyst. The chemical vapor deposition method can be a thermal chemical vapor deposition (TCVD) method, a plasma enhanced chemical vapor deposition (PECVD) method, a floating catalyst chemical vapor deposition (FCCVD) method and the like.

In the preparation process of the bionic microarray adhesive piece 2, a reactive plasma dry etching method can also be used as below: preparing a layer of polymer thin membrane with micro-scale thickness on a silicon chip, etching the aluminum membrane by an electron beam to form a micro-structure array, and performing dry etching by using oxygen plasma by means of great difference in etching rate of oxygen plasma between alumina and the polymer to transfer and reproduce the micro-structure on the aluminum membrane onto the polymer thin membrane.

In the preparation process of the bionic microarray adhesive piece 2, a soft etching method, a directed self-assembly method based on growth of micro/nano fine hair and the like can be used.

Preferably, the tubular portion or/and the ampulla of the internal covering membrane can be added with one or more longitudinal or oblique or crisscross or spiral or special-shaped ribs or a combination thereof by suturing, adhering, anchoring, weaving, hooking and clamping, riveting, thermoplastic treatment, freezing, gas pressing, electrostatic treatment and the like or/and a combination thereof or other known methods, and the ribs can reinforce, support, expand the internal covering membrane and prevent distortion thereof, and have other functions of realizing coordination with the internal covering membrane and realizing mutual support with the functions of the internal covering membrane.

Preferably, the ampulla 1 and the tubular portion 3 of the duodenal internal covering membrane can be closed up or folded into the shape of a ball, cylinder, capsule or spindle together in vitro, and the folding way can be as follows: the internal covering membrane is folded or curled or covered from the distal end to the proximal end and then the ampulla is centripetally turned inwards.

Preferably, the duodenal internal covering membrane can be sent into the duodenum via the upper digestive tract under the assistance of an endoscope and X-ray fluoroscopy or other medical or/and biological observation equipment and instruments, when the covering membrane is arranged, a multi-claw device (the number of claws can be matched with the precise arrangement of the bionic microarray adhesive piece) can perform centrifugal distraction on the ampulla 1 which is tuned inwards along the center via a duct of endoscopic forceps or/and other devices, further outwards turn and reset the ampulla 1 which is tuned inwards, position it in the duodenal bulb, adhere, and then softly push the distal end of the duodenal internal covering membrane to a target position by the device or/and gas or/and water or/and gravity or/and other methods. If a memory material is adopted, the covering membrane can be gradually spread out at memory temperature in the intestine, the ampulla 1 of the internal covering membrane is placed at the upper part of the duodenum, and the lower edge of the ampulla 1 is arranged on the side of the duodenal papilla and the auxiliary papilla (minor papilla) near the gastric pylorus and does not hinder liquid from the bile duct and the pancreatic duct from entering the intestinal cavity. The tubular portion 3 of the internal covering membrane is positioned at the duodenal descending part, the horizontal part and the ascending part following the upper part of the duodenum, and the extended tubular portion 3 is positioned in the jejunum section following the ascending part of the duodenum. When contents in the intestinal duct move, due to the absence of traction force which is nearly perpendicular, the duodenal internal covering membrane cannot be detached; and when the duodenal bulb expands, as the duodenal internal covering membrane has no opposite traction force which is nearly perpendicular, the duodenal internal covering membrane cannot be detached.

Preferably, when the duodenal internal covering membrane is recovered, a multi-claw device (the number of claws can be matched with the precise arrangement of the bionic microarray adhesive piece) can be inserted from the upper edge of the ampulla via the duct of the endoscopic forceps or/and other devices and easily realize detachment and recovery accordingly at the part forming an included angle of about 90° with the upper edge of the ampulla 1 by centripetal traction force which is near to the perpendicular direction, thereby avoiding avulsion and other injuries to the intestinal tissue. After detachment, the upper edge of the ampulla 1 is turned inwards immediately, then the detached bionic microarray adhesive piece 2 is accordingly adhered with other parts of the ampulla 1 itself, and then the duodenal internal covering membrane can be easily taken out and recovered.

Preferably, the duodenal internal covering membrane and the bionic microarray adhesive piece 2 thereof are soft, smooth, elastic and good in tissue compatibility, and have no acute systemic reaction, no chronic systemic reaction, no acute local reaction and no chronic local reaction.

The duodenal internal covering membrane diverts chyme and bile and pancreatic juice in vivo, avoids direct digestion, absorption and metabolism of gastric effluent in the duodenum, and can be prepared into a medical device for preventing and treating obesity and diabetes without injuring the intestinal tissue.

The length, the thickness, the elasticity and the shape of each part, the diameter-length ratio of the fine hair, the length of the fine hair, the diameter of the fine hair, the spacing interval between the fine hair and other parameters of the duodenal internal covering membrane are reference values, and can be specially designed according to needs in actual manufacturing.

### Example 1

A duodenal internal covering membrane can be made of a biocompatible biodegradable or non-biodegradable material or/and strongly hydrophobic material and mainly comprises a tubular portion 2 and a flared following ampulla 1, wherein bionic microarray adhesive piece 2 is adhered on the outer side of the ampulla 1.

The diameter, the length and the thickness of the tubular portion 3 can be matched with the duodenum and the jejunum in each person of different people groups. Preferably, the diameter is 25 mm, the length is matched with the duodenum and can extend to one section of the jejunum following the duodenum, the length is 500 mm, and the thickness of the internal covering membrane of the tubular portion 3 is 0.1 mm. The ampulla 1 is the flared part following the tubular portion 3. Preferably, the thickness of the internal covering membrane of the ampulla 1 is 0.1 mm, the flared following tubular portion 3 forms a progressive open acute angle, and the angle is preferably 45° C. Preferably, the upper edge of the ampulla 1 can be a wavy elastic membrane. Preferably, the bionic microarray adhesive piece 2 can be adhered to the ampulla 1 of the internal covering membrane, the precise arrangement can be rhombic, two rows or three rows can be adopted, and an adhesive can be biocompatible polyurethane, polyurethane, silicone resin, fluorinated ethylene propylene and the like or a combination thereof or other known materials and a combination thereof.

Preferably, the bionic microarray adhesive piece 2 can adopt an inductive coupling plasma (ICP) deep etching technology in a micro-electro-mechanical system (MEMS) to etch an erective microarray template on a silicon chip, cast polydimethoxysiloxane (PDMS) onto a silicon template pillar array, cure and then strip for demolding to obtain a polydimethoxysiloxane (PDMS) microarray hole template, cast liquid polyurethane or/and other biocompatible material onto the polydimethoxysiloxane (PDMS) micro-hole template, cure and demold to obtain a polyurethane bionic adhesion microarray. The invention does not exclude the use of other substances and other methods for preparing the adhesion microarray. A mussel-inspired adhesive protein polymer-dopamine-methacrylamide/methoxy ethyl acrylate copolymer (P(DMA-co-MEA)) is synthesized; and the synthesized dopamine-containing copolymer is dissolved in a dichloromethane solution, the polyurethane microarray is immersed in the solution, and a layer of dopamine-containing copolymer is modified on the outer surface of the polyurethane microarray. The invention does not exclude other substances (including modifying substances and modified substances) which can form strong adhesive force in drying conditions and also have strong adhesive force in water and other preparation methods. Preferably, the bionic microarray adhesive piece 2 have suitable contact surfaces, and the diameter-length ratio of the fine hair and the spacing interval between the fine hair are controlled for preventing mutual adhesion. Preferably, the duodenal internal covering membrane and the bionic microarray adhesive piece 2 thereof are soft, smooth, elastic and good in tissue compatibility, and have no acute systemic reaction, no chronic systemic reaction, no acute local reaction and no chronic local reaction.

Preferably, the tubular portion 3 of the internal covering membrane can be added with a spiral rib by an adhering or/and weaving method to reinforce, support, expand the internal covering membrane and prevent distortion thereof, and realize the function of realizing the coordination with the internal covering membrane.

### Example 2

Preferably, the ampulla 1 and the tubular portion 3 of the duodenal internal covering membrane can be closed up or folded into the shape of a cylinder together in vitro, and the folding way can be as follows: the internal covering membrane is folded or curled or covered from the distal end to the proximal end and then the ampulla 1 is centripetally turned inwards.

Preferably, the duodenal internal covering membrane can be sent into the duodenum via the upper digestive tract under the assistance of an endoscope and X-ray fluoroscopy, when the covering membrane is arranged, a multi-claw device (the number of claws is matched with the precise arrangement of the bionic microarray adhesive piece) can perform centrifugal distraction on the ampulla 1 which is tuned inwards along the center via a duct of endoscopic forceps, further outwards turn and reset the ampulla 1 which is tuned inwards, position it in the duodenal bulb, adhere, and then softly push the distal end of the duodenal internal covering membrane to a target position by the device or/and gas or/and water or/and gravity or/and other methods. When contents in the intestinal duct move, due to the absence of traction force which is nearly perpendicular, the duodenal internal covering membrane cannot be detached; and when the duodenal bulb expands, as the duodenal internal covering membrane has no opposite traction force which is nearly perpendicular, the duodenal internal covering membrane cannot be detached.

Preferably, when the duodenal internal covering membrane is recovered, the multi-claw device can be inserted from the upper edge of the ampulla 1 via the duct of the endoscopic forceps and easily realize detachment and recovery accordingly at the part forming an included angle of about 90° with the upper edge of the ampulla 1 by centripetal traction force which is near to the perpendicular direction. After detachment, the upper edge of the ampulla 1 is turned inwards immediately, then the detached bionic microarray adhesive piece 2 is accordingly adhered with other parts of the ampulla 1 itself, and then the duodenal internal covering membrane can be easily taken out and recovered.

### Example 3

The preparation of the bionic microarray adhesive piece 2 mainly comprises the following process steps: the first step: a glow discharge effect of CF4 gas is used for producing activation free radicals of F atoms. Then the activation free radicals of the F atoms can react with silicon or silicon dioxide to generate silicon tetrafluoride gas, thereby showing an etching effect. The second step: fluorine atoms are introduced into argon gas plasma, and fluorine and silicon can react fast by using the synergistic effect of the plasma, so that the etching effect is better. The third step: a mask plate pattern is introduced on a silicon chip, then an Oxford ICP180 etching system is used for etching a pillar array with high length-diameter ratio on the silicon chip, polydimethylsiloxane is finally cast onto the silicon template pillar array, then the silicon template pillar array is placed into an oven for baking and curing at 60° C. for 4 h, stripping and demolding are performed to obtain a polydimethylsiloxane hole array template, then other high polymer liquid is further cast onto the polydimethylsiloxane hole array template, and curing and demolding are performed to obtain a large-area micro-scale high polymer bionic foot setae adhesion array.

### Example 4

The tapered pointed end of a probe of an atomic force microscope is used to etch micro-holes on the surface of smooth paraffin wax, wherein the length of the holes is 3 μm, the aperture is 400 nm and the spacing interval between the holes is 1.5 μm; and a liquid polyimide material is cast into the holes, placing is performed for cooling, the paraffin wax is removed, and then micro-protuberances which are similar to a fine forked structure on gecko's setae are formed on the surface of the polymer after demolding.

### Example 5

An aluminum foil is placed into an acidic electrolyte, anodic oxidation is performed, the aperture and the spacing interval between the holes can be regulated and controlled by oxidation voltage and an acidic solution, the aperture is 150 nm, the length of the holes is 60 μm, and a bionic material can be polymethyl methacrylate and the like.

### Example 6

An electrostatic induction etching method is as follows: using a solution spin-coating method to prepare a layer of polymer thin membrane on a smooth silicon chip to serve as a lower electrode, taking another silicon chip as an upper electrode, retaining an air gap of 100-1000 nm between the surface of the polymer and the upper electrode, heating the polymer to be above glass transition temperature, applying direct current voltage of 30-40V to a capacitor, producing electric field intensity of 105V/m, forming a regular micro-structure and cooling to room temperature to obtain the corresponding polymer. If the upper electrode itself has the micro-structure, the polymer can accurately reproduce the same protuberant micro-structure.

### Example 7

A silicon template is passivated and etched with special gas, wherein a CRYO process and a BOSCH process can be adopted. The CRYO process is as follows: synchronously passivating and etching at low temperature below −100° C., wherein gas can be SF₆/O₂. The BOSCH process is as follows: separately etching and passivating at normal temperature, wherein etching gas can be SF₆, and passivating gas can be C₄/F₈. The aperture of prepared polystyrene is 200 nm, and the diameter-length ratio of fine hair is 1:10.

### Example 8

A photolithography technology (electron beam projection lithography, nano-imprint lithography and the like) can be used as below: drawing a mask which is tens or hundreds of times bigger than the actual size artificially or by a computer, scaling down to form an actual working template, attaching the template on a silicon substrate, and etching a bionic array shape which is the same with the template on the silicon substrate by a photon beam transmitting the template, or other etching technologies, such as an ion beam etching, are adopted for assistance. The fine hair with relatively large tail ends can be prepared from poly-p-xylene, and a layer of thin hydrophobic membrane is deposited on the surface to prevent mutual adhesion. The fine hair with concave surface can produce the maximum force of 18 N per cm², which is about 4 times higher than that of the fine hair with flat pointed ends. The adsorption force produced by the bionic material with such tail ends is about 70 times higher than that produced by the material with hemispherical tail ends.

### Example 9

The preparation of array nano-carbon tubes is as follows: decomposing gas containing a carbon element at high temperature by a chemical vapor deposition method, and enabling decomposed carbon atoms to generate an ordered carbon nanotube array directionally in a place with a catalyst. The chemical vapor deposition method can adopt a thermal chemical vapor deposition (TCVD) method, a plasma enhanced chemical vapor deposition (PECVD) method, a floating catalyst chemical vapor deposition (FCCVD) method and the like. The TCVD method can be used, Fe and Al are used as catalysts for growing the multi-wall carbon nanotube array with vertical precise arrangement on the silicon substrate in the atmosphere of ethylene and hydrogen at the temperature of 750° C. The length of the grown setae is 150-600 μm, the diameter is 200-800 μm, the adsorption force of 36 N can be produced per 1 cm², the adsorption force of the setae is 4 times of that of a gecko and 10 times of that of a common polymer material, and such ordered hierarchical structure can produce the adsorption force which is 4 times higher than a messy structure.

### Example 10

A reactive plasma dry etching method can be used as below: preparing a layer of polymer thin membrane with micro-scale thickness on a silicon chip, etching the aluminum membrane by an electron beam to form a micro-structure array, and performing dry etching by using oxygen plasma by means of great difference in etching rate of oxygen plasma between alumina and the polymer to transfer and reproduce the micro-structure on the aluminum membrane onto the polymer thin membrane. A soft etching method, a directed self-assembly method based on growth of micro/nano fine hair and the like can also be used.

### Example 11

A micro-adhesive array casting process can be used as below: manufacturing a thin plate micro-hole array by taking brass as a casting mold material and performing a known processing technology, and casting a micro-adhesive array in a vacuum environment, wherein a casting material is a biocompatible material, such as 184 type silicon rubber and the like; heating and curing, wherein a cured silicon rubber array matrix itself has no viscosity and has good self-cleaning property; and taking out a whole set of mold and the adhesive array, and cooling to normal temperature, wherein the temperature-variable demolding can be performed, a demolding agent can be liquid paraffin wax or dimethyl silicone oil, and a sealing agent can be solid paraffin wax. The array after demolding has the advantages of little strut fracture, good retention of the tail ends, reusability and the like. The adhesive array has the characteristics of anisotropic adhesive property, controllable normal adhesive force, relatively large tangential adhesive force and the like.

### Example 12

An erective micro-array template is etched on a silicon chip by using a ICP deep etching technology in MEMS, polydimethylsiloxane (PDMS) is cast onto the array, then curing, stripping and demolding are performed to obtain a polydimethylsiloxane (PDMS) template with micro-holes, then liquid polyurethane is cast onto polydimethylsiloxane (PDMS), and curing and demolding are performed to obtain a polyurethane bionic adhesive micro-array with the micro-array. (The main process steps are as follows: the first step: a glow discharge effect of CF4 gas is used for producing activation free radicals of F atoms. Then the activation free radicals of the F atoms can react with silicon or silicon dioxide to generate silicon tetrafluoride gas, thereby showing an etching effect. The second step: fluorine atoms are introduced into argon gas plasma, and fluorine and silicon can react fast by using the synergistic effect of the plasma, so that the etching effect is better. The third step: a mask plate pattern is introduced on a silicon chip, then an Oxford ICP180 etching system is used for etching a pillar array with high length-diameter ratio on the silicon chip, polydimethylsiloxane is finally cast onto the silicon template pillar array, then the silicon template pillar array is placed into an oven for baking and curing at 60° C. for 4 h, stripping and demolding are performed to obtain a polydimethylsiloxane hole array template, then other high polymer liquid is further cast onto the polydimethylsiloxane hole array template, and curing and demolding are performed to obtain a large-area micro-scale high polymer bionic foot setae adhesion array.) The invention does not exclude the use of other substances and other methods for preparing the adhesion microarray. A mussel-inspired adhesive protein polymer-dopamine-methacrylamide/methoxyethyl acrylate copolymer (P(DMA-co-MEA)) is synthesized; and the synthesized dopamine-containing copolymer is dissolved in a dichloromethane solution, the polyurethane microarray is immersed in the solution, and a layer of dopamine-containing copolymer is modified on the outer surface of the polyurethane microarray. The invention does not exclude other substances (including modifying substances and modified substances) which can form strong adhesive force in drying conditions and also have strong adhesive force in water and other preparation methods.

### Example 13

A rabbit dermal stem cell suspension with the density of 6×10⁴/ml is transferred into a culture dish, the culture dish is rotated to uniformly disperse cells to the surface, and culture is performed till the single layer is nearly converged; a culture medium is abandoned, the fresh culture medium is added, a test sample is placed at the center of the culture dish, trypsin is respectively dropped into the culture medium at 1 d, 2 d, 3 d, 4 d and 6 d to enable the cells adhered on the wall of the culture dish to fall into the culture medium, a definite quantity of the culture medium is taken and dropped onto a cell counting plate for counting growth of the cells; and at 6 d, the culture dish is placed under an inverted microscope for observing the growth of rabbit dermal stem cells, and the growth of the cells in an experimental group is good in the culture medium on the edge of the material, showing that the cells have been adhered on the edge of the material, and the cells are counted, wherein the concentration is 8.53×10⁴/ml. The cells in the culture dish at 1 d, 2 d, 3 d, 4 d and 6 d are counted, and a cell growth curve is drawn, prompting that the growth of the cells in the experimental group has no significant meaning with a normal control group (P<0.5).

### Example 14

A material leaching solution of the bionic microarray adhesive piece 2 is prepared according to part 12 of “national standard-biological evaluation of medical devices”, and the specific steps are as follows: selecting sterile physiological saline as a leaching medium, rinsing with tri-distilled water three times, irradiating with cobalt 60 for later use, and leaching the material at 37° C. for 72 h in a sterile state to prepare 100 ml of the leaching solution. 30 healthy male Balbc mice are randomly divided into three groups, namely a leaching solution group, a positive control group and a negative control group. Weighing, recording and labeling are performed immediately before beginning of testing. The leaching solution is shaken up, tail intravenous injection is performed on the Balbc mice according to 50 ml/kg, the physiological saline is used as negative control, 4.5 ml/L phenol water solution is used as positive control, and the reaction and the survival rate of the Balbc mice are observed within 72 h. The results show that the leaching solution group and the negative control group have no death, no obvious adverse reactions and no great significant meaning in changes in body weight.

### Example 15

The bionic microarray adhesive piece 2 is rinsed with tri-distilled water three times and irradiated with cobalt 60 for later use. 12 healthy adult New Zealand rabbits with one half of female rabbits and one half of male rabbits are randomly divided into three groups, intraperitoneal injection of 20% of urethan is performed for anesthesia, the hair in operation areas on the backs of the rabbits are cut off, the operation areas are disinfected with iodophor, the skin is cut open, subcutaneous tissues are separated, paraspinal muscles are exposed, the material thin piece is embedded and implanted at 30 mm to the midlines along the long axis of muscle fiber, sutured and disinfected, stitches are taken out after 1 w, the animals are killed at 1 w, 4 w and 12 w, the local paraspinal muscle tissues are taken, washed clean with physiological saline, washed with 4% paraformaldehyde and fixed in a 4% paraformaldehyde solution, and after 24 h, paraffin wax embedding, wax dipping and slicing are performed, HE dyeing is performed, and the observation is performed under an optical microscope. At 1 w after implantation, no obvious tissue edema occurs in the material group by naked-eye observation, non-obvious edema of striated muscles occurs in slices, the continuity of the muscle fiber is still permissible, little tissue interstitial inflammatory cell infiltration occurs, and little proliferation of the fiber tissues occurs on the edge of the tissue at the embedding part. At 12 w after implantation, no obvious tissue edema occurs in the material group, the structures of the tissues are still complete, no obvious inflammatory reaction occurs, and little wrapping of the fiber tissues occurs around the tissues at the embedding part.

### Example 16

A synthesized dopamine-containing copolymer is dissolved in dichloromethane, a polyurethane array is immersed in the solution, and a layer of dopamine-containing copolymer is modified on the outer surface of the array. Elastic modulus testing is performed by a nanoindenter, the resolution of force is 1 nN, the resolution of depth is 0.04 nm, the maximum load is 10 mN, the maximum pressing depth is 20 nm, a flat-head pressure head which is 100 μm long and 1 μm wide is selected, the load adopts a displacement control mode, the maximum vertical displacement of a probe is within 200 nm, and four points are tested for each sample. A bionic adhesive array after modification shows the adhesivity in water. The smaller the elastic modulus is, the greater the corresponding adhesivity is. The tangential adhesive force after modification can reach 2.21 N/cm², and the normal adhesive force can reach 2.15 N/cm². The adhesive force is increased along with the increases in pre-pressure, and when the pre-pressure is 6.11 N/cm², the adhesive force is maximum.

The length, the thickness, the diameter and the like of the invention are reference values and can be specifically designed according to individual needs in actual manufacturing.

The parts to which the invention does not relate contain the same contents with the prior art or can be implemented by adopting the prior art.

What is claimed is:

1. A duodenal internal covering membrane, wherein the covering membrane is made of a biocompatible biodegradable or non-biodegradable material and/or hydrophobic material and mainly comprises an elastic ampulla and a tubular portion, wherein the ampulla is positioned in the duodenal bulb, the tubular portion extends to the jejunum, the ampulla contains a biocompatible bionic microarray adhesive piece capable of realizing strong adhesion through a force exerting direction without pricking into an intestinal tissue and capable of being easily detached and recovered, the upper section of the duodenal internal covering membrane is a wavy, V-shaped, trapezoidal or crenellated ampulla elastic membrane, the ampulla attached with the bionic microarray adhesive piece is capable of performing telescopic or elastic motion by complying with the motion of the duodenum and the bulb as a whole, and the ampulla and the tubular portion is capable of being closed up or folded into the shape of a ball, cylinder, capsule or spindle together in vitro.

2. The duodenal internal covering membrane according to claim 1, wherein the bionic microarray adhesive piece is made of a biocompatible biodegradable or non-biodegradable material or/and hydrophobic material is capable of being selected from silicon rubber, polyurethane, multi-wall carbon nanotubes, polyester resin, polyimide, artificial rubber, epoxy resin, polydimethylsiloxane, polystyrene, polytetrafluoroethylene, Teflon, polydimethylsiloxane, poly-p-xylene, polyurethane, ethylene terephthalate, polymethylmethacrylate and the like or a combination and other suitable materials, the shape can be circular, oval, trapezoidal, square, triangular, cylindrical, rhombic, special-shaped and the like or a combination thereof, the size can be 1 nm² or above or a combination thereof, and the top end of adhesion fiber fine hair can be a curved (shovel-like) or flat pressing head-like or circular pressing head-like or layered structure or other shapes and structures and a combination thereof.

3. The duodenal internal covering membrane according to claim 1, wherein the bionic microarray adhesive piece is made of being adhered to the ampulla of the internal covering membrane by suturing, adhering, anchoring, weaving, hooking and clamping, riveting, thermoplastic treatment, freezing, gas pressing, electrostatic treatment and the like or/and a combination thereof or other methods and the like and a combination thereof, the precise arrangement can be circular, oval, trapezoidal, square, triangular, cylindrical, rhombic, special-shaped and the like or a combination thereof, one more than one row can be adopted, the bionic microarray adhesive piece can be arranged closely or separately or in other manners or arranged in a combination manner, and an adhesive can be biocompatible polyurethane, polyurethane, silicone resin, fluorinated ethylene propylene and the like or a combination thereof or other materials and a combination thereof.

4. The duodenal internal covering membrane according to claim 1, wherein the bionic microarray adhesive piece has a small area, low thickness and strong grasping force without pricking into the mucosa, the force exerting angle of flow of intestinal contents is difficult to realize detachment, the force exerting angle of a duct of endoscopic forceps is capable of easily realizing detachment, recovery and mounting, and adhesion and detachment can be performed repeatedly.

5. The duodenal internal covering membrane according to claim 1, wherein the bionic microarray adhesive piece is prepared by an atomic force microscope etching method, an alumina template hole injection forming method, other mold injection and electrostatic induction etching methods, an inductive coupling plasma etching technology, a photolithography technology (electron beam projection lithography, nano-imprint lithography and the like), array nano-carbon tubes, a reactive plasma dry etching method, a soft etching method, a directed self-assembly method based on growth of micro/nano fine hair and the like and a combination thereof or other methods.

6. The duodenal internal covering membrane according to claim 1, wherein the bionic microarray adhesive piece has great adhesive force, good stability, strong adaptability to material and appearance, good self-cleaning property, no injuries and pollution to the intestinal tissue and the like, and can realize mutual support with other parts in the functions.

7. The duodenal internal covering membrane according to claim 1, wherein the ampulla and the tubular portion of the duodenal internal covering membrane can be closed up or folded into the shape of a ball, cylinder, capsule or spindle together in vitro, and the folding way can be as follows: the internal covering membrane is folded or curled or covered from the distal end to the proximal end and then the ampulla is centripetally turned inwards.

8. The duodenal internal covering membrane according to claim 1, wherein the duodenal internal covering membrane can be sent into the duodenum via the upper digestive tract under the assistance of an endoscope and X-ray fluoroscopy or other medical or/and biological observation equipment and instruments, when the covering membrane is arranged, the covering membrane is subjected to centrifugal distraction along the center to outwards turn and reset the ampulla which is tuned inwards, position it in the duodenal bulb and adhere, when contents in the intestinal duct move, due to the absence of traction force which is nearly perpendicular, the duodenal internal covering membrane cannot be detached, and when the duodenal bulb expands, as the duodenal internal covering membrane has no opposite traction force which is nearly perpendicular, the duodenal internal covering membrane cannot be detached.

9. The duodenal internal covering membrane according to claim 1, wherein when the duodenal internal covering membrane is recovered, a multi-claw device (the number of claws can be matched with the precise arrangement of the bionic microarray adhesive piece) can be inserted from the upper edge of the ampulla via the duct of the endoscopic forceps and easily realize detachment accordingly at the part forming an included angle of about 90° with the upper edge of the ampulla by centripetal traction force which is near to the perpendicular direction, thereby avoiding avulsion and other injuries to the intestinal tissue; and after detachment, the upper edge of the ampulla is turned inwards immediately, then the detached bionic microarray adhesive piece is accordingly adhered with other parts of the ampulla itself, and then the duodenal internal covering membrane can be easily taken out.

10. The duodenal internal covering membrane according to claim 1, wherein the covering membrane can be prepared into a medical device for preventing and treating obesity and diabetes without injuring the intestinal tissue.
